(19) United States
(12) Patent Application Publication
(10) Pub. No.: US 2010/0298259 A1
(43) Pub. Date: Nov. 25, 2010

(54) PHARMACEUTICAL OR COSMETIC PREPARATIONS FOR TOPICAL AND/OR PARENTERAL APPLICATION, PREPARATION METHODS THEREOF AND USE OF SAME

(76) Inventors: Marc Moutet, Cachan (FR); Jean-Claude Yadan, Montreuil Sous Bois (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/599,510

(22) PCT Filed: Apr. 22, 2008
(86) PCT No.: PCT/FR08/50728

§ 371 Date: Aug. 6, 2010

(30) Foreign Application Priority Data
May 11, 2007 (FR) 0755034

(51) Int. Cl. A61K 31/728; A61P 17/02; A61Q 19/08; A61Q 19/00

(52) U.S. Cl. 514/53; 514/54

(57) ABSTRACT

The invention relates to a pharmaceutical or cosmetic composition for topical and/or parenteral application comprising, in a physiologically acceptable medium, at least one retinoid or salts and derivatives thereof, and at least one hyaluronic acid oligomer chosen from compounds containing from 1 to 6 disaccharide units. The invention is of use in particular in human dermatology or in reconstructive surgery.

The present invention relates to compositions for topical and/or parenteral application comprising, in a physiologically acceptable medium, a retinoid and/or salts thereof and/or derivatives thereof, and a hyaluronic acid oligomer constituted of 1 to 6 disaccharide units, to processes for the production of such compositions, and to uses thereof as pharmaceutical compositions, especially as a medicament, or as cosmetic compositions. Said compositions are for use in the treatment of dermatological conditions, in particular in the treatment by filling of wrinkles, fine lines, fibroblast depletions and any scars.

Skin aging is one of the most visible modifications of the process of senescence. In addition, the skin is exposed to many factors that accelerate this physiological process. A distinction can be made between two different types of skin aging. Firstly, intrinsic aging, that it is easier to evaluate on areas which are not normally exposed to the sun and, secondly, extrinsic aging, brought about by the interaction of environmental factors, in particular UV rays. These environmental factors have a much more marked effect on the parts of the body exposed to the sun, especially in individuals of light phototype. This is then also referred to as actinic aging. Other factors, such as dietary habits, smoking, excessive alcohol consumption, chronic diseases and endocrine gland dysfunctions, also contribute to this aging.

During intrinsic skin aging, the horny layer is relatively unmodified. The epidermis is atrophic and the dermal-epidermal junction is flattened, such that the adhesion to the dermis is weaker, facilitating the formation of bubbles. The thickness of the dermis is clearly reduced; there are fewer blood vessels. Fewer fibroblasts are also observed and their biosynthetic and proliferative capacities are reduced. The elastic fibres first undergo modifications, and subsequently disappear.

As regards extrinsic aging, an irregular, sometimes atrophic, sometimes hyperplasic, epidermis is observed, with signs of disorganization and of dysplasia. There are more melanocytes in certain areas, and fewer in others. The distribution of melanin in the epidermis is also irregular, subsequent to melanosome transfer problems. The number of Langerhans cells decreases. The small blood vessels are first dilated, and then become thinner and atrophy.

Wrinkles are the most visible signs of aging. A distinction can be made between several types, in particular superficial and deep wrinkles. Deep wrinkles are thought to be due to dermo-hypodermal modifications, whereas superficial wrinkles could be explained by dermal and possibly epidermal modifications. Wrinkles are especially due to the loss of elasticity of the skin. The effect on the subepidermal elastic network gives rise to superficial laxity of the aged skin and folding of its surface. The destruction of the elastic fibres in the reticular dermis is responsible for the loss of elasticity and of the skin's ability to return to its shape after stretching. A suitable treatment will be possible according to the type, the intensity and the topography.

The treatment of unattractive skin modifications related to aging has made enormous progress over the past few years.

A relatively large number of natural or synthetic substances have already been described as dermal implants, i.e. as substances injected directly into the skin, in order to remedy skin alterations resulting from aging, traumas or diseases.

Other therapeutic alternatives for these applications are in particular the local injection of deactivated botulinum toxin (Botox®) or the use of laser techniques. These various types of treatment are not exclusive and a combination thereof has even been recommended. Among the natural substances of human origin, collagen and hyaluronic acid are those which form the basis of the majority of products available on the market.

Hyaluronic acid is a ubiquitous natural polysaccharide which exists in the same form from the simplest bacterium to humans. It is a polysaccharide composed alternately of D-glucuronic acid and N-acetylglucosamine, linked to one another by alternating beta-1,4 and beta-1,3 glycosidic linkages. According to Saari H et al. Differential effects of reactive oxygen species on native synovial fluid and purified human umbilical cord hyaluronate. Inflammation 17 (1993):403-415, the polymers of this recurring unit may be between 10² and 10⁴ kDa in size, in vivo, hyaluronic acid taken from the umbilical cord having a weight of 2500 kDa.

Hyaluronic acid represents in particular a natural constituent of the dermis, where it plays an important role in the hydration and elasticity of the skin. However, it decreases in amount and in quality with age, leading to drying out of the skin, which becomes wrinkled. It is highly water-soluble and forms high-viscosity solutions in water. Because of these specific properties, hyaluronic acid is among the pharmaceutical products most commonly used.

However, in humans, hyaluronic acid is very rapidly eliminated from the plasma by degradation. Its plasma half-life after intravenous injection is very short, between 2.5 and 5.5 minutes, whereas in the skin, its half-life is from 0.5 to 2 days depending on its concentration. Its excretion in the urine is low, less than 1% of total clearance. In rabbits, the rate of elimination, in the skin, has been measured (Reed R K, Laurent U B, Fraser J R, Laurent T C. Removal rate of [3H]hyaluronan injected subcutaneously in rabbits. Am J Physiol. 1990 August; 259 (2 Pt 2): H532-5). It is nonexponential with a half-life of 0.5 to 1 day when its concentration is 5 mg/ml.

The tolerance of hyaluronic acid is very good and no immunogenicity has been associated with this substance. A very low incidence of side effects is thus observed.

The use of hyaluronic acid, alone or in combination, has thus been described for several medical applications, such as, for example, the treatment of osteoarthritis and also rheumatoid arthritis.

Injectable compositions such as, for example, hyaluronic acid alone, collagen alone or the combination of “hyaluronic acid and collagen” have already been used in repair surgery, in the context of the treatment by filling of wrinkles, fine lines, fibroblast depletions and any scars.

Currently, many dermal implants are used but none has yet been considered to be ideal in the context of a safe and healthy tissue augmentation (Naoum C, Dasiou-Plakida D. Dermal filler materials and botulin toxin Int J Dermatol. 2001 October; 40(10): 609-21).

However, because the bioavailability of hyaluronic acid is too low after injection and its injection frequency is too high, it cannot be used as such.

Of course, there has been an effort to develop compositions based on hyaluronic acid having a very good bioavailability and capable of more successfully withstanding the action of degradation enzymes. This makes it possible, in particular, to space out the procedures and to reduce the number thereof.

These compositions used as a dermal implant are all composed of stabilized hyaluronic acid and a large number of them comprise hyaluronic acid that has been chemically modified for this purpose. In addition, the hyaluronic acid included in these products is predominantly of nonhuman origin, for instance of avian or bacterial origin.

Numerous chemically modified hyaluronic acid derivatives in the form, in particular, of esters, amides and also derivatives having “intra- and/or interchain bridges” (crosslinked), are thus found in these compositions.

However, these modifications affect the physicochemical characteristics and the biological properties of hyaluronic acid, its potential immunogenicity and also its outcome after administration. These structural modifications of hyaluronic acid can lead to inflammatory reactions, as reported by Sopaar CNS, Patrinely J R Ophthalmic plastic and reconstructive surgery 2005 March; 21(2): 151-53.

Given the above, a problem that the invention is intended to solve is to produce compositions making it possible for hyaluronic acid to have a better bioavailability while at the same time conserving its physicochemical characteristics and its biological properties, and also a process for the production of such compositions.

As a solution to this stated problem, a first subject of the invention is a pharmaceutical or cosmetic composition, in particular for topical and/or parenteral application, comprising, as sole active ingredients, in a physiologically acceptable medium:
at least one compound chosen from retinoids, salts thereof and derivatives thereof, and
at least one hyaluronic acid oligomer chosen from compounds containing from 1 to 6 disaccharide units.

Preferably, the composition does not comprise any inhibitor of hyaluronic acid degradation.

The composition according to the invention in fact contains, as active ingredients, only the retinoid(s) and the hyaluronic acid oligomer(s); any other active ingredient is excluded.

A second subject of the invention is a process for the manufacture of such a pharmaceutical or cosmetic composition, comprising a step of mixing at least one compound chosen from retinoids, salts thereof and derivatives thereof, and at least one hyaluronic acid oligomer chosen from compounds containing from 1 to 6 disaccharide units with a physiologically acceptable medium. Preferably, the process according to the invention also comprises a step of preparing a physiologically acceptable medium, in which the active agents are mixed.

Finally, a third subject of the invention is the use of a composition according to the invention, for the manufacture of a medicament for use in the treatment and/or prevention of dermatological conditions.

A pharmaceutical or cosmetic composition according to the invention clearly increases the bioavailability of a hyaluronic acid, which is also included in the composition, or which is administered separately. The composition according to the invention makes it possible to space out the applications of hyaluronic acid and to reduce the number thereof and it is highly effective in filling wrinkles, fine lines, fibroblast depletions and any scars.

The applicant has demonstrated a decrease in hyaluronic acid catabolism in human keratinocytes, in vivo, to which hyaluronic acid, a hyaluronic acid oligomer chosen from compounds containing from 1 to 6 disaccharide units and a retinoid are applied, in the absence of an inhibitor of hyaluronic acid degradation.

Thus, surprisingly, the composition comprising a hyaluronic acid oligomer chosen from compounds containing from 1 to 6 disaccharide units and a retinoid confers better stability and better bioavailability on the hyaluronic acid also applied. Such a composition is more effective than the prior art compositions, and especially compositions comprising inhibitors of hyaluronic acid degradation, in filling wrinkles, fine lines, fibroblast depletions and any scars, and also when moisturizing the skin.

The invention will be understood more clearly upon reading the nonlimiting description which will follow.

The composition according to the invention comprises, in a physiologically acceptable medium, at least one retinoid and/or salts thereof and/or derivatives thereof and one hyaluronic acid oligomer chosen from compounds containing from 1 to 6 disaccharide units. In particular, it does not comprise any inhibitor of hyaluronic acid degradation.

The composition according to the invention may also comprise hyaluronic acid. Alternatively, the composition according to the invention may be administered to an individual to whom hyaluronic acid is administered independently. In this case, the hyaluronic acid may be included in a separate composition, which may be administered simultaneously or else at a different time to that of the administration of the composition according to the invention. The separate composition comprising hyaluronic acid may be administered topically, orally or parentally, for example by injection.

The term “physiologically acceptable medium” is intended to mean, according to the invention, a medium compatible with the skin and, optionally, with its appendages (eyelashes, nails, hair) and/or the mucous membranes.

In the compositions according to the invention, the retinoid and/or salts thereof and/or derivatives thereof, and the hyaluronic acid oligomer chosen from compounds containing from 1 to 6 disaccharide units and, if applicable, the hyaluronic acid, are present in proportions that can range from 0.0000001% to 10%, preferably from 0.00001% to 1% by weight, relative to the total weight of the composition. In the present description, and unless otherwise specified, it is understood that, when concentration ranges are given, they include the upper and lower limits of said range.

The compositions according to the invention comprise hyaluronic acid.

The term “hyaluronic acid” is intended to mean a ubiquitous natural polysaccharide which exists in the same form from the simplest bacterium to humans. It is a polysaccharide alternately composed of D-glucuronic acid and N-acetylglucosamine, linked to one another by alternating beta-1,4 and beta-1,3 glycosidic linkages. According to Saari H et al., Differential effects of reactive oxygen species on native synovial fluid and purified human umbilical cord hyaluronate. Inflammation 17 (1993):403-415, the polymers of this recurring unit may be between 10² and 10⁴ kDa in size, in vivo, hyaluronic acid taken from the umbilical cord having a weight of 2500 kDa.

Advantageously, the hyaluronic acid is natural.

The term “natural hyaluronic acid” is intended to mean a hyaluronic acid that is non-stabilized and non-chemically modified in the form, in particular, of esters or amides or in the form of derivatives having “intra- and/or interchain bridges” (crosslinked), such modifications affecting the physicochemical characteristics and the biological properties of said hyaluronic acid, and also what becomes of it after administration.

The compositions according to the invention comprise a retinoid and/or salts thereof and/or derivatives thereof, taken alone or as a mixture.

Among the retinoids that may be part of the compositions according to the invention, retinol, retinal, retinoic acid, adapalene or salts and derivatives thereof, taken alone or as a mixture, will preferably be chosen, more preferably retinol.

The term “retinoid salt” is intended to mean in particular an alkali metal salt, an alkaline-earth metal salt or an organic amine salt. The term “retinoid derivative” is intended to mean in particular the esters, such as retinyl palmitate, retinyl acetate, retinyl stearate, retinyl oleate, retinyl propionate or else retinyl linoleate.

Advantageously, the retinoids used in the compositions according to the invention are retinoids that exist naturally in the human body.

The term “oligosaccharide” is intended to mean polymers formed from a number n (with n less than or equal to 100) of monosaccharides by glycosidic linkage, in particular any oligosaccharide which limits the penetration of hyaluronic acid into the cells of the skin, these being in particular keratinocytes and fibroblasts.

Among the oligosaccharides, taken alone or as a mixture, which can be part of the compositions according to the invention, hyaluronic acid oligomers, preferably hyaluronic acid dimers to dodecamers, said dimer comprising one component hyaluronic acid disaccharide unit, and the dodecamer comprising six of these disaccharide units, more preferably hyaluronic acid tetramers to hexamers, more preferably the hyaluronic acid pentamer, will be chosen. The molecular weight of a hyaluronic acid disaccharide unit is approximately 400 Da. An oligomer of one to six hyaluronic acid disaccharide units therefore has a molecular weight of between 400 and 2400 Da.

The term “oligomer” is intended to mean, according to the IUPAC in Pure Appl. Chem., Vol. 68, No. 12, pp. 2287-2311, 1996, a molecule of intermediate molecular weight, the structure of which comprises a small quantity of molecules having a lower molecular weight. Reference is made to a molecule having an intermediate molecular weight, when the removal of one or of a few constituent units will significantly modify the properties of the molecule.

The hyaluronic acid oligomers, taken alone or as a mixture, which can be part of the compositions according to the invention, are hyaluronic acid dimers and tetramers, said dimer comprising one component hyaluronic acid disaccharide unit, and the tetramer comprising two of these disaccharide units. The molecular weight of a hyaluronic acid disaccharide unit is approximately 400 Da. An oligomer of one or two hyaluronic acid disaccharide units therefore has a molecular weight of, respectively, 400 to 800 Da.

Advantageously, the oligomers used in the compositions according to the invention are compounds that exist naturally in the human body.

In the compositions according to the invention, the oligomer is used at concentrations of between 10⁻⁹ M and 10⁻³ M, preferably between 10⁻⁹ M and 10⁻⁵ M.

The compositions according to the invention do not comprise any inhibitor of hyaluronic acid degradation.

The term “inhibitor of hyaluronic acid degradation” is intended to mean a compound capable of reducing, or even blocking, either the extracellular or the intracellular catabolism of hyaluronic acid, preferably a compound capable of reducing, or even blocking, the extracellular catabolism of hyaluronic acid, more preferably a compound capable of inhibiting the extracellular hyaluronidase present in the skin.

In a known manner, the compositions according to the invention may also contain the usual adjuvants known to those skilled in the art.

The compositions according to the invention can be formulated for topical and/or parenteral application.

The term “topical application” is intended to mean external application to the skin or the mucous membranes.

When they are for topical application, the compositions may be in any of the galenical forms normally used for topical administration. By way of nonlimiting example of topical compositions, mention may be made of compositions in liquid, pasty or solid form, and more particularly in the form of ointments, aqueous, aqueous-alcoholic or oily solutions, dispersions of the optionally two-phase lotion type, serum, aqueous, anhydrous or lipophilic gels, powders, impregnated pads, syndets, wipes, sprays, foams, sticks, shampoos, compresses, washing bases, emulsions of liquid or semiliquid consistency of the milk type, obtained by dispersion of a fatty phase in an aqueous phase (O/W) or vice versa (W/O), a microemulsion, suspensions or emulsions of soft, semiliquid or solid consistency of the white or coloured cream, gel or ointment type, suspensions of microspheres or nanospheres or lipid or polymeric vesicles, or microcapsules, microparticles or nanoparticles or polymeric or gelled patches for controlled release.

When they are for parenteral administration, the compositions according to the invention may be applied subcutaneously or intradermally. By way of nonlimiting example of parenteral compositions, mention may be made of compositions in the form of solutions or suspensions for perfusion or for injection.

By way of nonlimiting example given simply as an illustration and which can in no way limit the scope of the invention, hyaluronic acid may be administered in the form of an injectable aqueous solution and a composition according to the invention comprising a hyaluronic acid dimer is administered in the form of a cream.

In the context of a combined administration of hyaluronic acid and of a composition according to the invention, the administration frequencies may be identical or different.

Advantageously within the context of the invention, the frequency of administration of hyaluronic acid injected in the form of an injectable aqueous solution may range from 4 to 24 months, preferably from 4 to 16 months, whereas those of the composition according to the invention, administered topically, for example in the form of a cream, may range from 1 to 7 days, preferably from 1 to 3 days.

According to a specific embodiment of the invention, the process for the manufacture of a composition comprises the steps of preparing a physiologically acceptable medium and of mixing an effective amount of a hyaluronic acid tetramer.

The invention also relates to the use of at least one compound chosen from retinoids, salts thereof and derivatives thereof, and of at least one hyaluronic acid oligomer chosen from compounds containing from 1 to 6 disaccharide units, or of a composition as described above, for the manufacture of a medicament for use in the treatment, improvement and/or prevention of dermatological conditions.

More particularly, the invention relates to the use of at least one compound chosen from retinoids, salts thereof and derivatives thereof, and of at least one hyaluronic acid oligomer chosen from compounds containing from 1 to 6 disaccharide units, or of a composition as described above, for the manufacture of a cosmetic or pharmaceutical composition for use in the treatment, improvement and/or prevention of skin aging. The term “skin aging” is intended to mean wrinkles, fine lines, fibroblast depletions and scars. Such a composition is suitable for the treatment of wrinkled and/or aged skin, and aims in particular to prevent and/or reduce the effects thereof. The treatment of wrinkles, fine lines, fibroblast depletions and any scars is carried out in particular by filling.

In particular, the composition according to the invention may be applied to the areas of the face or of the forehead that are marked with expression wrinkles.

The invention also relates to the use of at least one compound chosen from retinoids, salts thereof and derivatives thereof, and of at least one hyaluronic acid oligomer chosen from compounds containing from 1 to 6 disaccharide units, or of a composition as described above, for the manufacture of a cosmetic or pharmaceutical composition for use in reconstructive surgery.

The present invention will now be illustrated by means of the following examples.

### EXAMPLE 1

### Composition No. 1

### Injectable Solution No. 1

This composition is prepared in a manner that is conventional for those skilled in the art:

| | |
|---|---|
| (NAC-Glucuronic acid)² tetramer | 0.002% |
| Retinol | 0.00001% |
| Water | qs 100% |

### EXAMPLE 2

### Composition No. 2

Injectable Solution No. 2 Containing Hyaluronic Acid, Coupled with a Cream According to the Invention

### Injectable Solution

| | |
|---|---|
| Hyaluronic acid | 2% |
| Water | qs 100% |

### Cream

| | |
|---|---|
| (NAC-Glucuronic acid)¹ dimer | 0.002% |
| Retinol | 0.00001% |
| Stearic acid | 3.00% |
| Mixture of glyceryl monostearate | 2.5% |
| and PEG stearate (100 EO) | |
| PEG stearate (20 EO) | 1.0% |
| Cyclopentadimethylsiloxane | 10.00% |
| Plant oils | 7.00% |
| Synthetic oils | 6.00% |
| Silicone gum | 0.20% |
| Stearyl alcohol | 1.00% |
| Water | qs 100% |

1-10. (canceled)

11. A pharmaceutical and/or cosmetic composition comprising, in a physiologically acceptable medium:
at least one compound chosen from retinoids and salts thereof, and
at least one oligosaccharide chosen from hyaluronic acid oligomers having from 1 to 6 disaccharide units,
wherein the at least one compound chosen from retinoids and salts thereof and the at least one oligosaccharide are the only active ingredients of the composition.

12. The pharmaceutical and/or cosmetic composition of claim 11, wherein the at least one compound chosen from retinoids and salts thereof is chosen from retinol, tretinoin, adapalene, retinal, retinyl palmitate, retinyl acetate, retinyl stearate, retinyl oleate, retinyl propionate retinyl linoleate, and salts thereof.

13. The pharmaceutical and/or cosmetic composition of claim 12, wherein the at least one compound chosen from retinoids and salts thereof is chosen from retinol, tretinoin, adapalene, and salts thereof.

14. The pharmaceutical and/or cosmetic composition of claim 11, wherein the at least one oligosaccharide is chosen from hyaluronic acid dimers and hyaluronic acid tetramers.

15. The pharmaceutical and/or cosmetic composition of claim 11, wherein the pharmaceutical and/or cosmetic composition is formulated for topical application.

16. The pharmaceutical and/or cosmetic composition of claim 11, wherein the pharmaceutical and/or cosmetic composition is formulated for parenteral application.

17. The pharmaceutical and/or cosmetic composition of claim 11, wherein the pharmaceutical and/or cosmetic composition is in a form chosen from a perfusable solution, an injectable solution, a perfusable suspension, or an injectable suspension.

18. The pharmaceutical and/or cosmetic composition of claim 11, wherein the pharmaceutical and/or cosmetic composition is in a form chosen from a dispersion of a fatty phase in an aqueous phase, or a dispersion of an aqueous phase in a fatty phase.

19. A method of making the pharmaceutical and/or cosmetic composition of claim 11, comprising mixing the at least one oligosaccharide and the at least one compound chosen from retinoids and salts thereof with a physiologically acceptable medium.

20. A method of treating a reconstructive surgery patient, comprising administering the pharmaceutical and/or cosmetic composition of claim 11 to the reconstructive surgery patient.

21. The method of claim 20, wherein the pharmaceutical and/or cosmetic composition is administered at the site of the reconstructive surgery.

22. The method of claim 20, wherein the pharmaceutical and/or cosmetic composition is administered orally.

23. A method of treating and/or preventing aging of skin of a subject, comprising administering to the subject a pharmaceutical and/or cosmetic composition comprising, in a physiologically acceptable medium:
at least one compound chosen from retinoids and salts thereof, and
at least one oligosaccharide chosen from hyaluronic acid oligomers having from 1 to 6 disaccharide units,
wherein the at least one compound chosen from retinoids and salts thereof and the at least one oligosaccharide are the only active ingredients of the composition.

24. The method of claim 23, wherein the method comprises administering the pharmaceutical and/or cosmetic composition topically.

25. The method of claim 23, wherein the method comprises administering the pharmaceutical and/or cosmetic composition parenterally, by injection, or by perfusion.

26. The method of claim 23, wherein the at least one compound chosen from retinoids and salts thereof is chosen from retinol, tretinoin, adapalene, and salts thereof.

27. A method of treatment by filling of wrinkles, fine lines, fibroblast depletions and scars, comprising administering to the subject a pharmaceutical and/or cosmetic composition comprising, in a physiologically acceptable medium:
at least one compound chosen from retinoids and salts thereof, and
at least one oligosaccharide chosen from hyaluronic acid oligomers having from 1 to 6 disaccharide units,
wherein the at least one compound chosen from retinoids and salts thereof and the at least one oligosaccharide are the only active ingredients of the composition.

28. The method of claim 27, wherein the pharmaceutical and/or cosmetic composition is applied to areas of at least one of the face or forehead of the subject, said areas containing wrinkles.

29. The method of claim 27, wherein the method comprises administering the pharmaceutical and/or cosmetic composition parenterally, by injection, or by perfusion.

30. The method of claim 27, wherein the at least one compound chosen from retinoids and salts thereof is chosen from retinol, tretinoin, adapalene, and salts thereof.
